(19) United States
(12) Patent Application Publication
(10) Pub. No.: US 2015/0273131 A1
(43) Pub. Date: Oct. 1, 2015

(54) PHOSPHATE AND UREA ADSORPTION FOR DIALYSIS

(71) Applicant: TRIOMED AB, LUND (SE)

(72) Inventors: Carin Malmborg, Hoor (SE); Nina Meinander, Sodra Sandby (SE)

(73) Assignee: TRIOMED AB, LUND (SE)

(21) Appl. No.: 14/646,784

(22) PCT Filed: Nov. 22, 2013
(86) PCT No.: PCT/SE2013/000184

§ 371 Date: May 22, 2015

(30) Foreign Application Priority Data
Nov. 23, 2012 (SE) 1230133-9

(51) Int. Cl. A61M 1/36; B01J 20/22

(52) U.S. Cl. CPC A61M 1/3679; B01J 20/226

(57) ABSTRACT

Claims 1-5 relate to methods for treatment of the human or animal body by therapy, since the claims describe changing the compositions of the circulation dialysis fluid, which will affect the ion concentration in the patient's blood. See PCT rule 43bis.1 (b) and PCT rule 67.1.(iv). Nevertheless, an examination has been conducted for these claims. The examination has been made in respect of the technical content of the claims.

### FIELD OF INVENTION

The present invention relates to a phosphate adsorbent for dialysis fluids for use in treatment of renal diseases.

### BACKGROUND

Patients suffering from renal diseases need dialysis treatment.

There are two different modalities of dialysis, namely hemodialysis and peritoneal dialysis. In hemodialysis, blood from the patient is circulated in an extracorporeal circuit into contact with one side of a membrane of a dialyzer, the other side being in contact with a dialysis fluid. Substances are transferred over the membrane via diffusion and convection. In peritoneal dialysis, the dialyzer membrane is in principle replaced by an endogenous membrane, namely the peritoneal membrane of the patient.

During dialysis, large quantities of dialysate are consumed. The spent dialysate is normally discarded.

In order to reduce the amount of used fluid, the spent dialysis fluid may be reused and regenerated by adsorption of certain substances by an adsorption column. This is called adsorption dialysis, which has been suggested more than 40 years ago.

Most adsorption columns use activated carbon for removal of many unwanted substances. However, activated carbon cannot efficiently adsorb phosphate or urea. In addition, activated carbon cannot adsorb certain electrolytes, such as sodium, potassium, magnesium or calcium, should that be required.

In order to adsorb urea, one previously used method is to pass the spent dialysate through a column comprising urease, which converts urea into ammonia and carbon dioxide or ammonium ions and carbonate ions. The ammonium is removed by for example zirconium phosphate. However, residual ammonium may be toxic to the patient and may increase the pH. Other methods of removing urea are highly desired.

Another substance that needs to be removed is phosphate, since otherwise hyperphosphataemia may develop, which is a common condition among patients with renal failure. Removal of phosphate through conventional dialysis is often not adequate, and blood phosphate levels may be further controlled by limiting dietary intake and by using oral phosphate binders.

Orally ingested calcium-containing compounds such as calcium carbonate may be used for controlling the level of serum phosphorus, but calcium accumulation often leads to hypercalcaemia with possible side effects including soft-tissue calcification, hypercalcaemic nephropathy, metabolic alkalosis, polyuria and constipation.

In an adsorbent dialysis system where dialysis fluid is regenerated and recirculated, phosphate needs to be continuously removed from the dialysis fluid in order to keep the concentration gradient of phosphate over the dialysis membrane high, and contribute to removal of phosphate from the patient's blood as efficiently as possible.

In addition to calcium carbonate, current clinically used oral phosphate binders include for example Sevelamer, a polyallylamine polymer, lanthanum carbonate and calcium acetate/potassium carbonate. Recently, chitosan was reported to decrease salivary and serum phosphate levels in hemodialysis patients, when chewed in the form of a chewing gum between meals, see patent publication WO 2006/061336 A2.

A Chitosan-iron (III) complex has been reported to bind phosphate both in vitro and in vivo, when given to rats orally (Baxter et al., J Pharm Pharmacol 2000, vol 52:863). However, the use of iron(III)-chitosan in human patients may not be feasible, as iron(III)-phosphate has been withdrawn from the list of allowed substances in food, in the European Union.

Thus, there is a need of a phosphate adsorbent, which avoids the problems mentioned above.

### SUMMARY OF THE INVENTION

Accordingly, an object of the present invention is to mitigate, alleviate or eliminate one or more of the above-identified deficiencies and disadvantages singly or in any combination.

In an aspect, there is provided a use of a chitosan adsorbent complexed with a metal ion for simultaneous adsorption of urea and phosphate from a dialysis fluid, wherein the metal ion is copper(II) bound to chitosan. The copper(II)-chitosan may be present in an amount of at least 15 g, which is sufficient to satisfy one quarter of the daily phosphate removal need from recirculated peritoneal dialysis fluid.

In an embodiment, the chitosan may be a low molecular weight chitosan with a viscosity of about 25 cP (0.025 Pas). In alternative embodiment, the chitosan may be a high molecular weight chitosan with a viscosity of about 1300 cP (1.3 Pas).

### BRIEF DESCRIPTION OF THE DRAWINGS

Further objects, features and advantages of the invention will become apparent from the following detailed description of embodiments of the invention with reference to the drawings, in which:

FIG. 1 is a structure scheme of chitosan molecules in complex with a copper ion, which can bind up to two molecules of urea.

### DETAILED DESCRIPTION OF EMBODIMENTS

Below, several embodiments of the invention will be described. These embodiments are described in illustrating purpose in order to enable a skilled person to carry out the invention and to disclose the best mode. However, such embodiments do not limit the scope of the invention. Moreover, certain combinations of features are shown and discussed. However, other combinations of the different features are possible within the scope of the invention.

In hemodialysis, the spent dialysate is normally discarded, resulting in consumption of large volumes of water of high purity (up to hundred liters per treatment of 4 hours). Often, reverse osmosis water is used, which is expensive to produce in large quantities. In addition, a reverse osmosis apparatus is cumbersome and takes up a large space and produces noise during use thereof.

In peritoneal dialysis, the peritoneal dialysis fluid is normally sterilized, for example by autoclaves. This procedure also adds to the costs and complexity.

In order to reduce the amount of dialysis fluid required, the dialysis fluid may be regenerated by passing the dialysis fluid through an adsorbent column or cartridge and reuse of the regenerated fluid. The adsorbent column most often comprises activated carbon, which is effective for removal of many undesired waste products or metabolite products from a dialysis fluid, including uric acid. However, activated carbon may not efficiently remove for example phosphate and urea.

Patients with renal failure develop hyperphosphatemia during long-term dialysis treatment, resulting in secondary hyperparathyroidism. Chronic accumulation of phosphate in dialysis patients may lead to highly increased serum concentrations of inorganic phosphorous of more than 6 mg/dL.

One reason for hyperphosphatemia in dialysis patients may be due to a reduced phosphate clearance by the dialysis membrane during hemodialysis. However, the dialysis liquid comprises no phosphate and thus the diffusive transport of phosphate over the membrane is at maximum during conventional hemodialysis. It is also observed that clearance of phosphate is largest during the first 30 minutes of hemodialysis and then is reduced to sometimes less than 50%. The difficulty of diffusive removal of phosphate by hemodialysis might depend on the fact that phosphate is present in the intracellular fluid and it takes time to transport the phosphate to the blood. Thus, there is a great rebound of blood concentrations of inorganic phosphorous as soon as the dialysis session is over.

In patent publication WO 2011/000086, it is suggested to use hemodialysis with a dialyzer having a membrane surface area of at least 3.0 m². It is stated that hemodialysis with such a large membrane reduces the phosphate levels not only during the dialysis treatments but also between the dialysis sessions. It is suggested that fluctuating serum phosphate in the intradialytic intervals at hemodialysis trice weekly may play an important role in the pathogenesis of hyperphosphatemia.

It has been reported that average dietary phosphate intake is 3.8 to 4.7 g/day, while only about 1 g phosphate per day can be eliminated by hemodialysis.

Phosphate removal by peritoneal dialysis may be more efficient, since peritoneal dialysis is performed more often and during longer times compared to hemodialysis, typically 4 hours, trice weekly. However, there are almost no reports that support such a theory.

In an attempt to reduce influx of phosphate, most patients need to consume oral phosphate binders. Calcium-based phosphate binders have traditionally been used for treatment of hyperphosphatemia. However recent data support an association between vascular calcification and accelerated cardiovascular disease in end-stage renal disease, and observational data suggesting that higher doses of calcium-based phosphate binders may contribute to vascular calcification. Furthermore, these medications appear to only modestly reduce serum phosphate levels and calcium phosphate product. Therefore, there has been considerable interest in controlling serum phosphate while minimizing oral calcium load. While most attention has focused on the use of non-calcium containing phosphate binders such as Sevelamer and lanthanum, modifying the dialysis regimen to improve phosphate clearance is an alternative approach that has received little attention to date.

Patent publication U.S. Pat. No. 4,213,859A discloses the use of an adsorbent for selective removal of phosphate, namely an organic cation exchanger charged with a metal ion whose phosphate is poorly soluble in water. As metal ions are mentioned: thorium, iron, tin lanthanum, aluminum and zirconium. All these metal ions form phosphates having a solubility of not higher than 10 mg/L in water. When a phosphate ion comes close to the metal ion on the cation exchanger, the phosphate ion reacts with said metal ion and is removed from the dialysis fluid.

The metal ions may be immobilized at other carriers than a cation exchanger. In an article by Baxter et al., J Pharm Pharmacol 2000, vol 52:863, “Effect of Iron(III) Chitosan Intake on the Reduction of Serum Phosphorus in Rats” the iron (III)-chitosan complex was reported to bind phosphate both in vitro and in vivo, when given to rats orally (Baxter et al., J Pharm Pharmacol 2000, vol 52:863).

Without being bound by any theory, it is believed that phosphate ions are attached or complexed to the iron in the iron(III)-chitosan to thereby be removed from the fluid. However, exposure to iron(III)-phosphate might be hazardous to humans. In fact, the substance is not allowed to be included in food in the European Union. Iron(III)-phosphate was withdrawn from the list of allowed substances in the directive 2002/46/EC in 2007.

Dialysate also comprises urea, which should be removed. One promising adsorbent for removing urea from body-fluids is copper(II)-chitosan, as suggested in an article: “Preparation and Characterization of Chitosan/Cu(II) Affinity Membrane for Urea Adsorption”, by Jiahao Liu, Xin Chen, Zhengzhong Shao, Ping Zhou, published in Journal of Applied Polymer Science, Vol. 90, 1108-1112 (2003). A urea adsorption of up to about 80 mg urea per gram chitosan (8%) was reported.

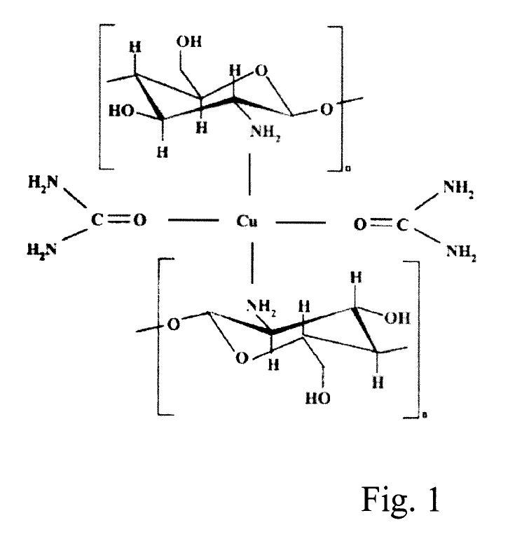

As shown in the article, there is produced a porous copper(II)-chitosan membrane, wherein copper ions are complexed to the chitosan polymer amine groups as shown in FIG. 1.

Each copper ion can complex with from one or up to four, but preferably two amine groups of the chitosan polymer, and when two groups from separate chitosan polymer chains are bound by copper, cross-linking and stabilization of the porous membrane is achieved.

Additionally, phosphate may be adsorbed by the chitosan membrane, but the exact mechanism for adsorption is unknown at present.

We have unexpectedly found that copper(II)-chitosan has the same or higher phosphate binding capacity compared to iron(III)-chitosan or lanthanum(III)-chitosan in a solution comprising both phosphate and urea, such as a dialysis solution, which should be regenerated.

Without being bound by any theory, it is suggested that phosphate may bind to copper(II)-chitosan by another mechanism than direct metal ion binding.

In iron(III)-chitosan, both urea and phosphate may bind to the iron(III) atoms, which have been immobilized on chitosan by amine bonds. In this case, urea and phosphate would compete for the same binding site, and the phosphate-binding ability of iron(III)-chitosan saturated with urea would decrease.

Contrary to this, copper(II)-chitosan adsorbs urea via binding to the copper-atom, while phosphate may also have other binding mechanisms. In this case, there would be no competition between urea and phosphate for the same binding site and copper(II)-chitosan may adsorb phosphate also after it has been more or less saturated by urea. This is an important advantage during adsorption dialysis, since the urea adsorbent, in this case copper(II)-chitosan, will be saturated with urea relatively fast, because of the large amounts of urea to be removed.

Thus, the copper(II)-chitosan amount can be dimensioned to remove the desired amount of urea, for example 15 g/day, and will at the same time adsorb and remove a considerable amount of phosphate at no expense or extra amount of adsorbent.

Based on the above discovery, we have found that metal-complexed chitosan, where the metal component is iron(III), lanthanum(III) or copper(II) had a significantly higher phosphate binding capacity compared to uncomplexed chitosan. Unexpectedly, the highest phosphate binding capacity is achieved with copper(II)-chitosan. This is unexpected because both iron(III) and lanthanum(III) have been or are being used clinically as oral phosphate binders and lanthanum(III) is used for removal of excess phosphate from polluted lake water. However, the use of copper(II)-chitosan as a particularly efficient phosphate binder, has not previously been reported. Additionally, metal-complexed chitosan also binds urea. Binding of urea to chitosan complexed with other metal ions than copper has not been previously described.

Copper(II), iron(III) or lanthanum(III) can be complexed with untreated chitosan or with macroporous chitosan membranes, which are prepared as described in the article “Control of pore size in macroporous chitosan and chitin membranes”, by Xianfang Zeng and Eli Ruckenstein, published in Industrial and Engineering Chemistry Research, vol. 35, 4169-4175 (1996). The metal ion is provided as a soluble salt, either inorganic or organic, such as CuSO₄, CuCl₂, CuBr₂, CuF₂, Cu(NO₃)₂, Cu-acetate, Cu-citrate, Cu-lactate, Cu-oxalate, Cu-propionate, Cu-benzoate, Cu-succinate, Cu-malonate or Cu-stearate; Fe₂(SO₄)₃, FeCl₃, Fe(NO₃)₃, Fe-citrate; LaCl₃, LaBr₃, LaF₃, LaI₃, La(BrO₃)₃, La(NO₃)₃, La₂(SeO₄)₃, La₂(SO₄)₃, La-acetate or La-oxalate.

Fe(III) or La(III) or Cu(II) can be bound to chitosan macroporous membranes by incubating the membrane in a solution of the metal salt, such as Copper acetate, CuCl₂, LaCl₃ or Fe₂(SO₄)₃ for 2-24 hours, followed by thorough washing of the membranes with water. The metal ion can be bound onto unprocessed chitosan powder by incubating chitosan powder in a solution of the metal salt, such as Copper acetate, CuCl₂, LaCl₃ or Fe₂(SO₄)₃ for 2-24 hours, followed by thorough washing with water.

The resulting metal-chitosan membranes or powder efficiently bind phosphate as well as urea from aqueous solutions with a composition similar to that of peritoneal dialysis fluid, and can be used as phosphate and/or urea adsorbent in a regeneration system of dialysis fluid for recirculation. Based on the specific phosphate binding capacity of 0.45 mmole per gram Cu(II)-chitosan porous membranes (see example 1), 15 g (dry weight) of membranes can bind an amount of phosphate corresponding to one quarter of the total daily phosphate removal need from recirculated peritoneal dialysate (estimated to about 25 mmole per day).

The following examples show the experiments carried out.

### EXAMPLES OF PREPARATION OF METAL ION BOUND CHITOSAN POWDER AND EVALUATION OF PHOSPHATE BINDING CAPACITY

### Example 1

Chitosan powder from two different sources was used: 1) Chitoclear chitosan (Primex) produced from shrimp shell with a viscosity of 1300 cP (1.3 Pas) (indicating relatively high molecular weight) and 2) Medical grade chitosan (Biotech Surindo) produced from crab shell with a viscosity of 23.8 cP (0.0238 Pas) (indicating a relatively low molecular weight). The viscosity is measured by dissolving 1% chitosan in 1% acetic acid. 0.5 g of chitosan powder was suspended in 50 mL of 100 mM solution of 1) copper acetate, 2) CuCl₂ 3) Fe₂(SO₄)₃, 4) LaCl₃ and 5) in pure water. The suspensions were incubated on an orbital shaker overnight. The chitosan was filtered and washed several times with water in a Büchner funnel with suction. The washed chitosan was incubated in room temperature in 50 mL of a dialysis fluid containing: 92 mM NaCl, 1.75 mM CaCl₂.2H₂O, 0.25 mM MgCl₂.6H₂O, 85 mM glucose, 40 mM sodium lactate, 21 mM urea and 5 mM NaH₂PO₄ at pH 7.4 for 1 hour. Samples of the dialysis fluid were taken before and after incubation and analyzed for phosphate concentration. Phosphate binding capacity was calculated based on the originally weighed amount of dry chitosan powder and is presented in Table 1.

TABLE 1
Phosphate binding capacity of chitosan powder complexed with different
metal ions.

| | Metal salt | Phosphate binding |
|---|---|---|
| Chitosan | treatment | (mmole/g) |
| Primex | Copper acetate | 0.45 |
| Primex | CuCl₂ | 0.41 |
| Primex | Fe₂(SO₄)₃ | 0.25 |
| Primex | LaCl₃ | 0.08 |
| Primex | none | 0.02 |
| Biotech Surindo | Copper acetate | 0.45 |
| Biotech Surindo | CuCl₂ | 0.43 |
| Biotech Surindo | Fe₂(SO₄)₃ | 0.31 |
| Biotech Surindo | LaCl₃ | 0.1 |
| Biotech Surindo | none | 0.03 |

### Example 2

Different forms of Cu-chitosan; gel beads made from different suppliers of chitosan and porous membranes made from Surindo chitosan, fibers from Hismer, and powder from Chitoclear were investigated for phosphate adsorption.

| | | | | | | initial | final | | Result |
|---|---|---|---|---|---|---|---|---|---|
| | | | | weight | volume | conc. | conc. | | phosphate |
| Sample | chitosan | form | copper | g | ml | mM | mM | Solution | mmole/g |
| 1 | surindo | gel beads | acetate | 2.0 | 50 | 0.8 | 0.01 | PD | 0.02 |
| 2 | chitoclear | gel beads | acetate | 2.0 | 50 | 0.8 | 0.02 | PD | 0.02 |
| 3 | surindo | membrane | acetate | 3.8 | 60 | 1.6 | 0.06 | PD | 0.02 |
| 4 | chitoclear | powder | chloride | 2.0 | 200 | 4.8 | 0.7 | water | 0.4 |
| 5 | Hismer | fiber | sulphate | 0.8 | 100 | 4.8 | 0.9 | water | 0.5 |

PD = by a patient used peritoneal dialysis solution

1) Surindo gel beads: 6% chitosan was dissolved in an aqueous solution of 4% acetic acid and stirred for 1.5 hours, the solution wad added drop wise into an aqueous solution of 2.5% sodium hydroxide through a glass pasteur pipette. The mixture is kept under stirring overnight. The beads were washed until neutral pH and then treated with 0.1M CuAc for 3 hours, and washed thoroughly again and dried and grinded.

2) Chitoclear gel beads: 2% chitosan was dissolved in an aqueous solution of 2% acetic acid and stirred for 30 minutes. The solution wad added drop wise into an aqueous solution of 2.5% sodium hydroxide through a glass pasteur pipette. The mixture was kept under stirring overnight. The beads were washed until neutral pH and dried and grinded. Then the powder was treated with 0.1 M CuAc for 4 hours and washed thoroughly again and dried and grinded.

3) Surindo membrane: 3% chitosan was dissolved in an aqueous solution of 2.8% acetic acid. A quantity of silica particles corresponding to 8 times of the amount of chitosan was added and stirred during 2 hours. The suspension was poured in petri dishes of 9 cm diameter, 15 g per dish, and dried in a fume hood. When dried the silica was dissolved in an aqueous solution of 5% sodium hydroxide overnight, and the membranes was washed until neutral pH, and then dried and grinded. Then the powder was treated with 0.1M CuAc for 4 hours and washed thoroughly again and dried and grinded.

4) Chitoclear chitosan flakes were used as received, and put in a copper chloride aqueous solution of 0.05M for 4 hours. The sample was washed, dried and grinded.

5) A sample of Hismer fibers were soaked in a copper sulfate aqueous solution of 0.025M during 8 hours. The sample was washed thoroughly and then dried.

Samples 1-3 were soaked in by a patient used peritoneal dialysis solution (PD). The phosphate concentration was not sufficient in the PD samples, since all phosphate was removed by the chitosan and the maximum capacity was probably not reached. For that reason we prepared stronger phosphate solution in water, pH were adjusted to 6.5-7.4 (sample 4-5) according to the table above. During 4 hours the samples were stirred/shaken in room temperature.

The best adsorption capacity result was obtained from the fiber sample, as much as 0.5 mmole/g Cu-chitosan, but also the powder showed potential with the adsorption capacity of 0.4 mmole/g Cu-chitosan. Gel beads and membrane adsorption capacities are also mentioned although they did not show up their maximum capacity due to a too low phosphate content in the PD solution.

All Cu-chitosans adsorbed phosphate well leaving very small concentrations in the solution after adsorption until they reached the maximum capacity.

Chloride is a negative ion common in human, just as phosphate. For that reason. we investigated the possibility that Cu-chitosan may adsorb chloride. The result was that Cu(II)-chitosan did not adsorb any substantial amount of chloride, but at least 75% of the phosphate.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Although the present invention has been described above with reference to specific embodiment and experiments, it is not intended to be limited to the specific form set forth herein. Rather, the invention is limited only by the accompanying claims and, other embodiments than those specified above are equally possible within the scope of these appended claims.

1. A method of using a chitosan adsorbent complexed with a metal ion for simultaneous adsorption of urea and phosphate from a dialysis fluid, wherein the metal ion is copper(II) bound to chitosan.

2. The method according to claim 1, wherein the chitosan is a low molecular weight chitosan with a viscosity of about 25 cP (0.025 Pas).

3. The method according to claim 1, wherein the chitosan is a high molecular weight chitosan with a viscosity of about 1300 cP (1.3 Pas).

4. The method according to claim 1, wherein said copper(II)-chitosan is present in an amount of 15 g, which is sufficient to satisfy one quarter of the daily phosphate removal need from recirculated peritoneal dialysis fluid.

5. A method for simultaneous adsorption of urea and phosphate from a dialysis fluid, wherein the dialysis fluid is passed through a material comprising a chitosan adsorbent complexed with a metal ion, in which the metal ion is copper(II) bound to chitosan.
